(19) United States
(12) Patent Application Publication
(10) Pub. No.: US 2008/0027505 A1
(43) Pub. Date: Jan. 31, 2008

(54) SYSTEM AND METHOD FOR TREATMENT OF HEADACHES

(75) Inventors: Howard Levin, Teaneck, NJ (US); Mark Gelfand, New York, NY (US)

Correspondence Address: NIXON & VANDERHYE, PC, 901 NORTH GLEBE ROAD, 11TH FLOOR, ARLINGTON, VA, 22203, US

(73) Assignee: G&L CONSULTING, LLC, New York, NY (US)

(21) Appl. No.: 11/828,731

(22) Filed: Jul. 26, 2007

Related U.S. Application Data
(60) Provisional application No. 60/820,347, filed on Jul. 26, 2006.
(60) Provisional application No. 60/826,850, filed on Sep. 25, 2006.

(51) Int. Cl. A61N 1/34

(52) U.S. Cl. 607/46

(57) ABSTRACT

A method and apparatus for treatment of cervicogenic headaches by transvascular application of stimulation energy to nerves in the neck and head. A catheter equipped with electrodes is inserted into a vertebral or occipital vein in proximity to peripheral nerves that conduct pain signals. An external to the body or implanted generator is used to apply stimulation energy to the targeted nerves.

### RELATED APPLICATION

This application claims the benefit of the U.S. Patent Provisional Utility Application Ser. No. 60/820,347, entitled “Transcatheter Occipital Denervation System and Method” filed Jul. 26, 2006 (NV 4343-34) and U.S. Patent Provisional Utility Application Ser. No. 60/826,850 entitled “Transvenous Nerve Stimulation for Cervicogenic Pain” filed Sep. 25, 2006 (NV 4343-38), both of which applications are incorporated by reference herein in their entirety.

### BACKGROUND OF THE INVENTION

This invention relates to a method for treatment of headaches by neuromodulation of peripheral nerves. More particularly, the present invention relates to methods and apparatus for achieving modulation, denervation and stimulation of nerves that conduct headache pain such as occipital nerves and other nerves via transvascular application of energy such as electric field energy and pulsed electric field. Both long term implantable pulse generators with implanted leads and external generators with temporary catheters are disclosed.

It is estimated that up to 40 million people in the United States suffer from chronic headaches. Most of these people do not consult doctors because they consider the problem to be too trivial or they think that no treatment is available. Patients who do consult a physician are usually those whose headaches significantly disrupt their lives. Over 2 million people in the United States experience transformed migraine—frequent headaches that have features of both migraine and tension headaches. These headaches are chronic daily or almost-daily occurrences, and usually last more than 4 hours. Transformed migraine often results in medication overuse, and the severity and progression are disabling and refractory to treatment. Cluster headaches are the most intense headaches of all, leading some patient to thoughts of suicide. Headaches occur in clusters, frequently during the same season each year, with each episode lasting for several weeks or months. The pain often wakes the patient from sleep—sometimes at the same time—every night and usually lasts for 30 to 90 minutes. Such regular occurrence, however, is not always present. The pain is described as retro-orbital, unilateral and is associated with agitation, nasal congestion, conjunctival injection and lacrimation.

Cervicogenic Headaches:

As its name suggests, the pain is referred from a primary source in the neck. It is believed that convergence of afferents from cervical nerves C1-C3 with trigeminal afferents, at the level of second order neurons, results in the perception of headache consequent to cervical irritation. Diagnostic blocks that have been used in cervicogenic headache include blocks of the greater occipital nerve (GON), lesser occipital nerve (LON), cervical zygapophysial joints and their nerve supply, atlantoaxial and atlanto-occipital joints, cervical nerve roots and epidural steroid injections. Cervicogenic headaches are very common in elderly patients due to arthritic changes in the cervical spine. Pain described as radiating from the neck or occipital in location suggests this diagnosis. Pain of cervical spine origin, however, can sometimes be felt in the front of the head. Loss of sensation over the occipital area, often on one side can accompany occipital neuralgia. If the headache is occipital and has a burning or lancinating quality, greater occipital neuralgia is the likely cause. Blockade of the occipital nerve by a local anesthetic is relatively easy to perform and may provide lasting relief. Many types of headaches including cluster and migraine will sometimes respond to occipital blocks as well. The prevalence of cervicogenic headache in the general population is estimated to be 0.4%-2.5%, but is as high as 20% in patients with chronic headache.

Occipital Neuralgia:

Occipital neuralgia is a term used to describe a cycle of pain-spasm-pain originating from the suboccipital area (base) of the skull that often radiates to the back, front, and side of the head, as well as behind the eyes, pairs of nerves that originate in the area of the second and third vertebrae of the neck. While most people's nerve roots originate in similar places on the spine, cadaver studies show a wide variety of differences between individuals as to the course of the nerves once they leave the spinal column. Often the nerves follow a curving course that passes through various muscles in the upper back, neck and head. These nerves supply areas of the skin along the base of the skull and partially behind the ear. While the occipital nerves do not directly connect with structures within the skull itself, they do interconnect with other nerves outside of the skull and form a continuous neural network that can affect any given area through which any of the main nerves or their branch fibers pass.

Occipital neuralgia is often defined as a paroxysmal jabbing pain in the distribution of the greater or lesser occipital nerves. It is characterized by pain in the cervical and posterior areas of the head that may/may not radiate to the sides of the head as well as into the facial and frontal areas. It may occur as the nerves exit the trapezii or splenius muscle groups. Compression of these nerves may result in a burning dysasthesias in the occiput with or without radiation behind the ear. Nerve compression can occur from cervical degeneration or post-traumatic compression of the cervical vertebrae C2 or C3 nerves. The clinical features of the condition are pain and sensory change in the distribution of the relevant nerve, localized nerve trunk tenderness.

Treatments for occipital neuralgia ranges from rest, heat, massage, exercise, antidepressants, nerve blocks, neurectomy, cervical rhizotomy, surgical release of the occipital nerve within the trapezius to neurolysis of the great occipital nerve with or without section of the inferior oblique muscle. Recently, there has been increased interest in subcutaneous electrical stimulation of the occipital nerve for the treatment of occipital neuralgia. The neurotechnology market for treating chronic migraine and other forms of headache pain has moved closer to commercial viability as several research institutions and manufacturers in the U.S. and Europe have made progress in occipital nerve stimulation (ONS). The market in many ways seems to mirror the advent of the market for spinal cord stimulation (SCS) systems to treat chronic back pain, and indeed, some SCS vendors are looking to use their same implantable pulse generators (IPGs) for ONS, albeit with leads directed to more anterior placements in the C1-C3 region at the top of the neck.

Neuroablation for Treatment of Pain:

Many surgical and interventional procedures have been undertaken in an attempt to alleviate chronic pain and most involve ablation or excision of central or peripheral neural tissue. Neuroablation, or destruction, of neural tissue has been the mainstay of surgical procedures for chronic pain for many past years. The effectiveness of these therapies was facilitated by diagnostic nerve blocks that allowed short term relief of pain and relatively precise location of nerves that conduct pain. In that regard, it seems clear that it is the diagnostic part that limits the success of invasive pain therapies. Since nerves can not be visualized, recent progress in imaging did not benefit pain therapies to the same degree as, for example, interventional cardiology and oncology. Moreover, the recognition that nerve injury is often the cause of chronic pain syndromes has resulted in reluctance to induce further nerve damage in non-terminal patients.

Radiofrequency Lesioning:

Radiofrequency (RF) lesioning is a common, proven means of treating chronic pain. Continuous radiofrequency current is used to heat a small volume of nerve tissue, thereby disrupting pain signals from that specific area. RF therapy uses a needle electrode to conduct current that destroys tissue with high temperature. In pain management, the goal of destructive RF lesioning (also called ablation) therapy is to selectively destroy nerve tissue to stop pain signals. For this to happen, the temperature of targeted nerve tissue must be at least 65 degrees Centigrade. For example RF energy of 500-1000 kHz, 15-50 W, 100-800 J, 30-75 V rms and 0.1-1 A rms, for 10-60 sec. can be used to destroy tissue extending several mm from the electrode by heating it to 65-100 degree Centigrade. Ideally, this procedure has a selective effect on nerve fibers, reducing pain in target areas, but leaving other sensory capabilities intact. In reality, this therapy can be rather unpredictable.

Pulsed Electric Field (Pulsed RF):

Another common treatment option for pain is pulsed RF therapy. In contrast to RF lesioning, pulsed RF delivers shorts bursts of RF current, instead of a continuous RF flow. This allows the tissue to cool slightly between each burst, significantly reducing the risk of destroying nearby tissue. Because pulsed RF therapy does not rely on heat to destroy nerves conducting pain, doctors can use this method to treat a wider range of painful areas, including peripheral nerves and near critical structures.

Although exact mechanism by which Pulsed RF disables nerves and prevents nerve conduction is unknown, there is a preponderance of evidence that it is effective. Intravascular application of Pulsed RF to disable renal nerves (controlling kidney function) is described in great detail in Published U.S. Patent Applications listed below. These applications show several ways of constructing a catheter that can be applied from the inside of a blood vessel to disable nerves proximal to the vessel, without damaging both the vessel and the nerves. These applications include:

U.S. Patent Publication 2006-0142801 Methods and apparatus for intravascularly-induced neuromodulation.

U.S. Patent Publication 2006-0041277 Methods and apparatus for renal neuromodulation.

U.S. Patent Publication 2005-0288730 Methods and apparatus for renal neuromodulation.

These applications do not disclose the use of intravascularly-induced pulsed RF to treat Cervicogenic pain or the use of occipital veins or arteries to bring the intravascular neuromodulation devices into proximity with an occipital nerve. Pulsed RF generators for treatment of pain are available from several vendors such as Valleylab Inc. a division of Tyco Healthcare Group LP (Boulder, Colo.). Construction and principals of operation of such generators are well known by persons of ordinary skill in this art and do not require detailed disclosure in this application.

### BRIEF DESCRIPTION OF INVENTION

While considerable progress has been made in treatment of headaches that do not respond to pharmacologic treatment, there remains a need to make these therapies less invasive, more targeted, less neuro-destructive and less expensive. In the case of cervicogenic headaches including occipital neuralgia and some types of migraines prior therapies required destruction of nerve tissue or placement of complex implantable neurostimulators. Progress of these therapies was impeded by the complex and variable anatomy of occipital nerves. Since nerves are not visible on X-ray, sophisticated imaging equipment was nearly useless for these therapies.

Neuromodulation by Application of Heat or Cold:

Some potential embodiments of the invention may include methods and apparatus for transvascular neuromodulation via thermal heating and/or thermal cooling mechanisms that are known and have been previously described for other neuromodulation applications other than headache pain control. Many embodiments of such methods and apparatus may reduce the targeted nerve activity. Thermally-induced (via heating and/or cooling) neuromodulation may be achieved via catheter apparatus positioned proximate target neural fibers, such as being positioned within adjacent vasculature (i.e., positioned intravascularly particularly inside an adjacent vein). Thermal neuromodulation by heating or cooling may be caused by directly effecting or otherwise altering the neural structures that are subject to the thermal stress.

As used herein, thermal heating mechanisms for neuromodulation include both thermal ablation and non-ablative thermal injury or damage (e.g., via sustained heating or resistive heating). Thermal heating mechanisms may include raising the temperature of target neural fibers above a desired threshold to achieve non-ablative thermal injury, or above a higher temperature) to achieve ablative thermal injury such as with application of radio frequency (RF) energy field. As used herein, thermal cooling mechanisms for neuromodulation also include non-freezing thermal slowing of nerve conduction and/or non-freezing thermal nerve injury, as well as freezing thermal nerve injury. Thermal cooling mechanisms may include reducing the temperature of target neural fibers below about zero degrees Celsius to achieve freezing thermal injury. Thermo ablation of conductive tissue with a cooling catheter is known and practiced in cardiology to treat cardiac arrhythmias.

In some embodiments, thermally-induced neuromodulation may be achieved by directly applying thermal cooling or heating energy to the target neural fibers. For example, a chilled or heated fluid can be applied at least proximate to the target neural fiber, or heated or cooled elements (e.g., a thermoelectric element or a resistive heating element) can be placed in the vicinity of the neural fibers. In other embodiments, thermally-induced neuromodulation may be achieved via indirect generation and/or application of the thermal energy to the target neural fibers, such as through application of a ‘thermal’ electric field, high-intensity focused ultrasound, laser irradiation, or other suitable energy modalities to the target neural fibers. For example, thermally-induced neuromodulation may be achieved via delivery of a pulsed or continuous thermal electric field to the target neural fibers, the electric field being of sufficient magnitude and/or duration to thermally induce the neuromodulation in the target fibers (e.g., to heat or thermally ablate or necrose the fibers). Additional and alternative methods and apparatus may be utilized to achieve thermally-induced neuromodulation, as described hereinafter. It is understood that for the purpose of this invention application of energy inside a vein can cause thrombosis and closure of the vein. For this invention it may be acceptable since the veins described in this invention are not vital and can be sacrificed.

Applicants set up following goals for a novel therapy of Cervicogenic Headaches:

1. To temporarily or permanently disable selected peripheral nerves in the neck or in the back of the head (occipital area).

2. To perform the therapeutic procedure without surgery that requires dissection of tissue or direct visualization of nerves or surgical implantation of stimulating electrodes that can damage nerves.

3. To achieve better (more precise and less time consuming) placement of electrodes for neuromodulation or denervation than is currently available with transcutaneous needle electrodes or surgery.

4. To implant pulse generators connected to implanted electrode leads placed in the veins of the neck and the head of the patient in proximity to peripheral nerves implicated in conduction of cervicogenic pain.

These goals do not exclude each other and do not need to be all achieved in one embodiment of the invention. It is understood that there are many different ways of disabling a nerve using transvascular catheters. Some examples of such devices and methods are known and even used in commerce.

Cervicogenic Headaches and Nerves in the Neck

As its name suggests, the pain is referred from a primary source in the neck. It is believed that convergence of afferents from cervical nerves C1-C3 with trigeminal afferents, at the level of second order neurons, results in the perception of headache consequent to cervical irritation. Diagnostic blocks that have been used in cervicogenic headache include blocks of the greater occipital nerve (GON), lesser occipital nerve (LON), cervical zygapophysial joints and their nerve supply, atlantoaxial and atlanto-occipital joints, cervical nerve roots and epidural steroid injections.

Cervicogenic headaches are very common in elderly patients due to arthritic changes in the cervical spine. Pain described as radiating from the neck or occipital in location suggests this diagnosis. Pain of cervical spine origin, however, can sometimes be felt in the front of the head. Loss of sensation over the occipital area, often on one side can accompany occipital neuralgia. If the headache is occipital and has a burning or lancinating quality, greater occipital neuralgia is the likely cause. Blockade of the occipital nerve by a local anesthetic is relatively easy to perform and may provide lasting relief. Many types of headaches including cluster and migraine will sometimes respond to occipital blocks as well. The prevalence of cervicogenic headache in the general population is estimated to be 0.4%-2.5%, but is as high as 20% in patients with chronic headache.

Disorders of the spinal joints, which include facet joints, have been implicated more commonly than disc herniation, attributing some 50% of spinal pain to these joints. According to American Society of Interventional Pain Physicians (ASIPP) guidelines the existence of lumbar and cervical facet joint pain is supported by a preponderance of scientific evidence. The prevalence of facet joint mediated pain in patients with chronic spinal pain has been established as 15% to 45% in low back pain, and 54% to 60% in neck pain utilizing controlled diagnostic blocks. The blockade of the dorsal rami of C2 and C3; in particular, the superficial medial branch of C3 also known as the third occipital nerve. This is particularly advantageous because the C2-C3 zygapophyseal joint is frequently a source of cervicogenic headache and is innervated by these nerves.

Limitations of Existing Implantable Neuromodulation Devices to Treat Pain

Spinal cord stimulation SCS systems and implantable intrathecal infusion devices are frequently used in managing chronic intractable pain. Notably and inevitably SCS and pumps in the clinical guidelines are placed the last after failure of all therapies including surgery and RF ablation. It seems that the reason is not as much their cost (SCS is considered cost effective) but high real or perceived risk and low long term effectiveness.

Present-day spinal cord stimulation (SCS) began shortly after Melzak and Wall proposed the (then plausible but now considered incorrect or incomplete) gate control theory in 1965. The gate theory proposes that activating large, myelinated afferent nerve fibers will affect the dorsal horn and inhibit transmission in small, unmyelinated primary afferent nerve fibers. Strategically placed epidural electrodes stimulate the dorsal columns to inhibit or modulate incoming nociceptive input through the smaller fibers. As a direct result of the gate theory, in 1967, Shealy et al implanted the first spinal cord stimulator device for the treatment of chronic pain. Over the course of the last 35 years, advancements in basic science research, and technology have led spinal cord stimulation to be an accepted, reliable treatment for many neuropathic and/or vascular insufficiency pain states. The mechanism of action of spinal cord stimulation is not completely understood. Despite what is known about the mechanism of action of SCS and the outcomes of many studies; much confusion remains regarding the indications for SCS.

Review of the literature demonstrates positive results in neuropathic and vascular insufficiency pain states. There is, however, no credible evidence to support the use of SCS in primarily nociceptive pain conditions (degenerative disc disease, sacroiliac dysfunction, arthritis, cancer, and acute tissue injury). In the United States, the primary indications for spinal cord stimulation are failed back surgery syndrome and complex regional pain syndromes type I and type II. However, in Europe, most interest in spinal cord stimulation has been in the treatment of chronic intractable angina and pain and disability due to peripheral vascular disease.

While randomized controlled trials in this field are almost non-existent, in the field of spinal cord stimulation (SCS), as with other interventional techniques in chronic pain management, there are numerous retrospective studies that promote the efficacy of spinal cord stimulation, showing approximately 60% efficacy that lasts approximately two years. Several authors reviewed the current literature regarding the treatment of chronic pain in failed back surgery patients with spinal cord stimulation. Most authors agree that 50% to 60% of patients with failed back surgery syndrome reported greater than 50% pain relief with the use of spinal cord stimulation. In addition to the declining success rate, complications also are common. These were predominantly electrode related problems i.e., migration, fracture, etc. Infection was less common, even though it was reported in 5% of the patients in 20 trials. From various studies wound infection occurs in 5% of cases, and 3% of implants require removal. Epidural abscess or hematoma may require surgical decompression. Electrode migration occurs in 35% of cases, and 23% require re-implantation. Electrode fracture occurs in 5%, and discomfort at the implantation site occurs in 13%. Other complications include pocket infections, a foreign body immune response causing allergic phenomena either local to the implant or systemically, and CSF leakage.

Amazingly, despite the limited evidence on SCS efficacy because of the lack of controlled studies, the use of spinal stimulation as a method of pain relief has increased exponentially during the last decade. Detailed figures on current SCS implantation rates are not readily available. In 1995 it was estimated that 14,000 stimulators were implanted world-wide and in Europe 5,000 units were implanted per annum by 1997.

Subcutaneous neurostimulation (SNS) where electrodes are implanted under the skin can be used to treat a variety of conditions, including occipital or transformed migraine, cervicogenic pain, V1 facial pain, failed peripheral nerve surgery, cluneal nerve pain, and stump pain. Spinal cord stimulation (SCS) systems and implantable intrathecal infusion devices are frequently used in managing chronic intractable pain. The mechanism of SNS and SCS is not completely understood. SNS and SCS both have known limitations. SCS shows approximately 60% efficacy that lasts approximately two years. In addition to the declining success rate, complications also are common. These were predominantly electrode related problems i.e., migration, fracture, etc. Electrode migration occurs in 35% of cases, and 23% require re-implantation.

Peripheral Nerve Stimulation (PNS) began as silicone cuff electrodes that were placed around the affected peripheral nerves and attached to a subcutaneous RF receiver. The technology has since been refined to multi array percutaneous wire electrodes, with power sources that range from RF receivers in combination with IPGs seemingly used off-label. The current techniques for PNS are minimally invasive (at least in theory). Electrodes can generally be placed during an outpatient procedure, with local anesthesia and sedation. One essential step toward effective use of neurostimulation in potential patients is a trial of the system through percutaneous lead placement. The purpose of the trial is to determine the effectiveness of the stimulation for relieving pain and improving the patient's quality of life. If this temporary placement of the stimulation system provides sufficient analgesia (often measured as >50% pain relief), allows the patient to sleep better, and uses less pain medication, then permanent placement of the system is considered.

A flat-paddle array is the most frequently used surgical lead, which incorporates a mesh apron to facilitate anchoring into the surrounding tissue. When implanting leads adjacent to peripheral nerves, intravenous sedation and local anesthesia are usually well tolerated and leave the patient alert enough to respond to intraoperative stimulation.

The electrode is placed by exposing a 5-cm segment of peripheral nerve proximal to the injury site and free of surrounding tissues. Nearby facial tissue or a facial graft is used to create a flap that covers the electrode to avoid direct contact with the nerve. The electrode lead is longitudinally inserted under the dissected section of the nerve, making sure that all electrode contacts remain close to the nerve. Once the electrode is placed, temporary electrode stimulation can be used to confirm proper lead position. As in SCS, the distal electrode wiring can be kept external so that there may be a prolonged screening stimulation before permanent implantation.

Power sources are most commonly situated subcutaneously in the anterior chest or abdominal wall, midaxillary midthoracic region, or posterior superior buttock region. Power sources for PNS in the lower extremities can be placed in the lateral thigh or extended into the abdomen. If appropriate, the electrode and the power source can be implanted by tunneling the lead extension wire to the receiver/generator pocket. The voltage requirements for PNS are generally much lower than for SCS, ranging from 0.2 to 3.0 V. Pulse widths range from 120 to 400 ms, with a frequency of 40-100 Hz. However, some pain is frequency-dependent, and this often requires frequencies of 1000 Hz.

Use of PNS has been limited in the past in some patients by the need for extensive surgical dissection in the affected region. However, the more current percutaneous electrode-placement techniques developed for SCS may make this less of an issue. Simple percutaneous perineural electrodes can be placed parallel to a major peripheral nerve quickly and easily, making more extensive nerve-dissection surgery unnecessary. This has been reported effective in treating failed carpal tunnel syndrome and failed ulnar transposition in which the nerve segment in the midforearm or the midhumerus.

Applicants propose a novel method to temporary block one or more of the peripheral nerves that conduct headache pain using electric energy such as a pulsed electric field, e.g., a pulsed radio frequency field (pulsed RF). This method is believed to overcome the limitations of prior therapies by a novel use of vertebral and occipital blood vessels (veins and arteries) to navigate, position and anchor catheter based instruments into the proximity of the occipital nerves. Applicants propose a novel method to temporary block one or more of the peripheral nerves that conduct headache pain using electric energy supplied continuously by an implanted pulse generator.

Occipital Nerve Modulation:

Pulsed RF is applied to block pain without creating a tissue lesion. It is typically performed by puncturing the skin of a patient with conductive metal needles (electrodes) and applying current from an RF generator to the needles. Pulsed RF therapies do not require meticulous placement of electrodes and tend to be faster and easier than thermal RF therapy. Pulse RF may tend not to destruct nerves because it does not create a lesion. Generally, to destroy soft tissue with radiofrequency, the RF must generate primary or secondary heat that denatures protein. It is believed that loss of nerve function occurs at 60 to 65 degrees Centigrade. Higher temperatures, e.g., higher than 65 degrees Centigrade, may be applied by RF to block transmission of nerve signals entirely. However, pulse RF typically raises tissue temperatures to about 42 degrees Centigrade, which does not result in substantial tissue injury.

Applicants propose a Pulsed RF therapy in which the therapy is adapted to treat Cervicogenic headaches. The therapy comprises introducing an intravascular catheter equipped with electrodes into an occipital vessel (occipital artery or vein). For example, left and right occipital veins drain the left and right back of the scalp into the corresponding left and right jugular veins. In their tortuous course these veins cross the occipital nerve. By gradually advancing the catheter into the veins, using common interventional radiology techniques assisted by, for example, X-ray fluoroscopy, the catheter can be positioned in the veins so that it is proximate to the occipital nerves. By periodically applying pulsed RF to the distal catheter end electrodes, the occipital nerves can be disabled, such as temporarily for weeks or months, and achieve long lasting pain relief without the risk of surgery.

The pulsed RF catheter may be equipped with multiple pairs of electrodes spaced along the catheter shaft. RF pulses may be applied to the pairs of electrodes simultaneously or sequentially to increase the probability of disabling an occipital nerve that conducts pain. The catheter may be equipped with a thermocouple to prevent excessive heating of blood and tissue. A signal from the thermocouple indicates the catheter temperature. A controller for the pulsed RF catheter monitors the thermocouple signal to ensure that the blood and catheter temperature and/or rate of temperature rise do not exceed threshold temperature and/or rate settings.

Pulsed RF parameters can include, but are not limited to, field strength, pulse width, the shape of the pulse, the catheter tip temperature, the number of pulses in a burst, number of bursts and/or the interval between pulses and bursts of pulses (e.g., duty cycle). Further, pulse may be a pulse burst of, for example two to ten pulses within a short duration, such as one second. The pulses within each burst may have varying amplitudes, such as for example between 90 and 400 Volts (peak), variable pulse rate, such as for example 1, 2, 3, 4, 5, 6, 7, 8 Hz (Pulse Per Second) and variable Burst Duration such as for example 10, 20, 30 ms. Suitable intervals between individual pulses or pulse bursts include, for example, intervals less than about 10 seconds and greater than three seconds. A suitable catheter target temperature can be for example 40 to 42 degrees Centigrade. An exemplary maximum temperature of the blood adjacent the catheter (as sensed by the thermocouple) may be 45 to 55 degrees Centigrade and an exemplary maximum temperature rise of blood adjacent the catheter may be 0.1 to 0.5 degree Centigrade per second for temperatures above 37 degrees Centigrade (which is approximately body temperature).

Alternatively other forms of thermal and non-thermal energy can be applied to nerve tissue via a catheter to modulate nerves. These include thermal energy (heating and cooling) RF energy destructive nerve tissue and others known in the field. Alternatively relatively continuous trains of electric pulses can be applied by implanted pulse generators to implanted leads positioned in targeted veins of the patient.

Cervicogenic Headaches:

Applicants propose treatment of cervicogenic headaches by directly stimulating selected spinal nervous tissue associated with cervicogenic headaches without limitations of current therapies such as Spinal Cord Stimulation.

In general, spinal nervous tissue (for example, a nerve roots) progresses from that within the epidural space to spinal ganglia, which exits the vertebral column, to a nerve plexus outside the vertebral column and, finally, to a more distal peripheral portion of the targeted nerve. A stimulation lead may be positioned so that its electrode position will span some portion of the selected nervous tissue spinal nervous tissue (i.e. epidural spinal nervous tissue, dorsal rami, spinal ganglion, neural plexus, and peripheral nerves), provided that the stimulation lead includes an adequate number of electrodes (for example, four or eight electrodes). Electrodes are positioned in the desired anatomic region proximal to the targeted nerve tissue by positioning the stimulation lead inside the vertebral vein or occipital vein (in case of peripheral occipital nerves). Electrode thus positioned is not likely to migrate and can be placed avoiding both surgery and invasion of the spinal epidural space. For example, electrodes positioned in the vertebral vein can be instrumental in stimulating the dorsal rami of C2 and C3 vertebrae that are known to conduct cervicogenic pain. Similarly, occipital veins are known to overlap occipital nerves implicated in cervicogenic headaches.

One embodiment of the therapy comprises introducing an intravascular lead equipped with electrodes into an occipital or into a vertebral vein. For example, left and right occipital veins drain the left and right back of the scalp into the corresponding left and right jugular veins. In their tortuous course these veins cross the occipital nerve. By gradually advancing the lead into the veins, using common interventional radiology techniques assisted by, for example, X-ray fluoroscopy, the lead can be positioned in the veins so that it is proximate to the occipital nerves. Lead advancement can be by following vascular routs: from Superior vena cava or Subclavian vein to Brachiocephalic vein to Subclavian to Vertebral vein and from External jugular vein to Posterior arcuate vein to Occipital vein. After the leads are secured in the desired position using X-ray landmarks, by periodically applying bipolar electric pulses to the selected pairs of lead electrodes, targeted nerves such as dorsal rami or occipital nerves can be stimulated.

The following illustrations and embodiments schematically depict the targeted peripheral veins that are suitable foe electrode placement and the peripheral nerve tissue that is a stimulation target. One illustration embodiment below shows implantation of an IPG and a lead with eight electrodes in the vertebral vein.

### BRIEF DESCRIPTION OF THE DRAWINGS

A preferred embodiment and best mode of the invention is illustrated in the attached drawings that are described as follows:

FIG. 1 illustrates an exemplary treatment of occipital nerves with a transvascular pulsed RF catheter inserted into an occipital vein.

FIGS. 2 and 3 illustrate a schematic showings of exemplary designs of catheters for transvascular application of energy to adjacent nerves.

FIG. 4 illustrates exemplary advantageous positions of proximity between occipital nerves and occipital arteries suitable for application of therapy.

FIG. 5 illustrates long term implantable embodiment of an IPG and a lead with electrodes in the vertebral vein.

FIG. 6 illustrates long term implantable embodiment of an IPG and a lead with electrodes in the occipital vein

### DETAILED DESCRIPTION OF THE INVENTION

This disclosure shows a proposed clinical use in which a RF catheter and controller/generator are applied to treat headaches by transvascular application of pulsed RF energy to occipital nerves. It further shows implantable embodiments of the invention where an implantable pulse generator is connected to at least one transvenous lead placed in a vein in the neck or back of the head of a patient. Specific positions in the venous system advantageous from anatomic stand point (easy to access) and suitable for treatment of cervicogenic pain are disclosed.

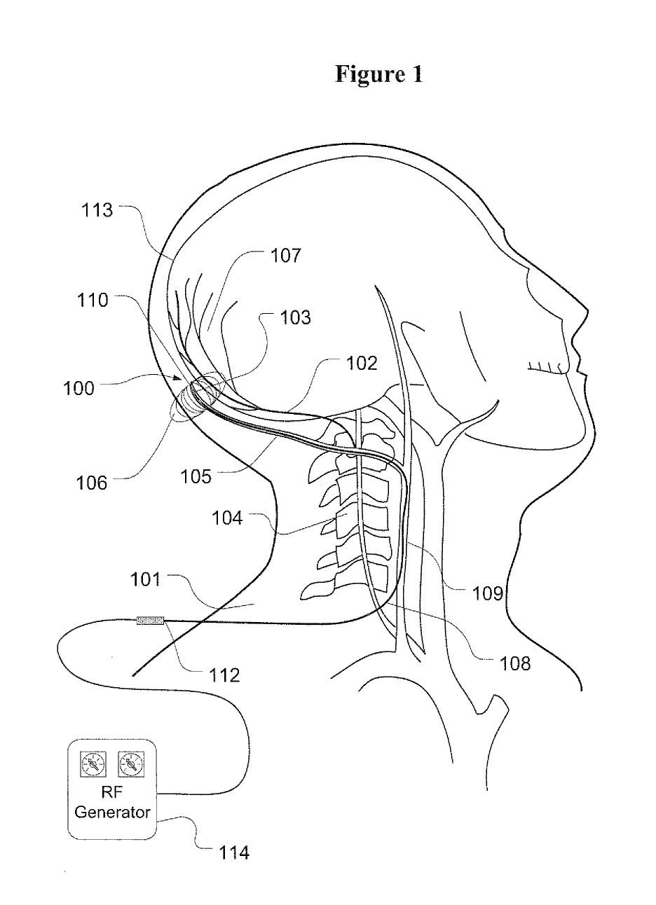

FIG. 1 illustrates a patient 101 treated with one embodiment of an RF pulsed catheter system 100 for transvascular denervation of occipital nerves. The catheter 108 is inserted by percutaneous puncture into the external jugular vein 109. The proximal end of the catheter 112 may be connected to an RF energy generator and controller 114. The generator/controller may include electronics to: generate a controlled pulsed signal to be transmitted by distal end 110 of the catheter as an RF pulsed signal; provide user input controls, e.g., display screen and keypad, to enter therapy conditions such as duration of pulse period and select pulse regime (the pulse regimes may be stored electronically in the controller and set by a manufacturer or source of software for the controller); emit alarms indicating an excessive temperature of blood heated by the catheter (which condition may also cause an automatic cessation of the RF pulse regime), and monitor sensors, such as a thermocouple in the catheter or impedance signal from electrodes. An electric connection (wired and/or wireless) between the generator and controller 114 and distal end of the catheter can include signal wires to conduct RF energy and/or temperature signals.

To position the catheter in the patient and after the catheter tip has been inserted into the jugular vein, the distal end 110 of the catheter 108 is advanced into the occipital vein 105 that branches from the jugular vein 109. The movement of the catheter may be accomplished using well known catheter advancement techniques. For example, well known interventional radiology techniques, e.g. including X-ray fluoroscopy, are available to advance a catheter through the jugular vein and into the occipital vein and then through the occipital vein to a region proximate to an occipital nerve.

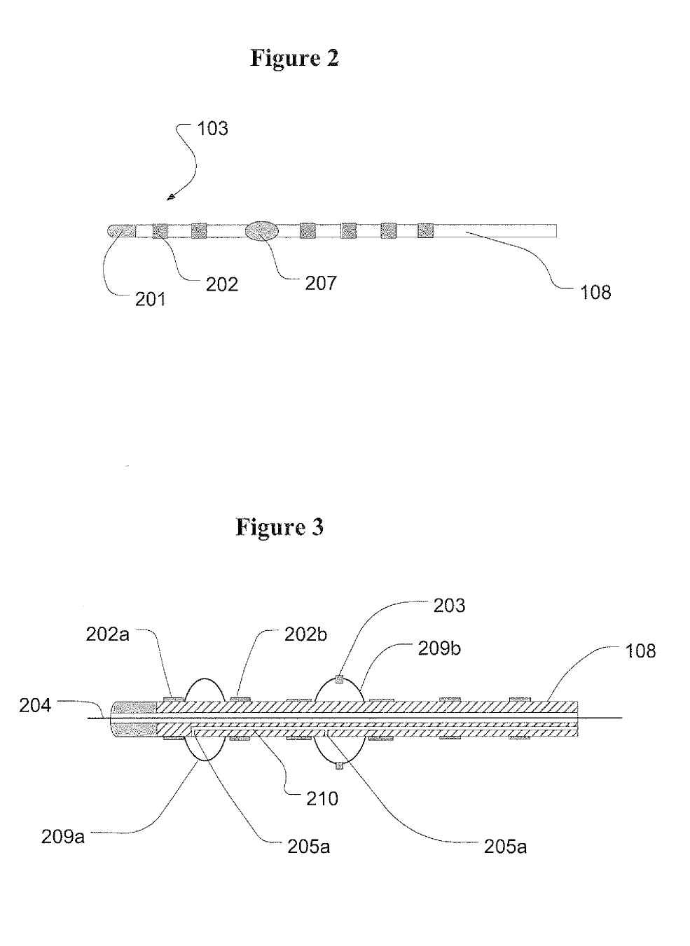

The distal end 110 of the catheter includes electrodes, such as surface electrodes which may be metallic ring collars made of gold or platinum or other metal alloy commonly used to manufacture catheter electrodes typically used for ablation of tissue embedded in the surface of catheter and coupled by individual wires to the controller/generator 114. Alternatively, electrodes may, for example, be fabricated in a form of spirally wound coils of metal wire. Wound coils allow the catheter to be more flexible in navigating blood vessels. An RF energy field 106 is released from the distal tip electrodes 103 (there can be 2, 4, 8 or more electrodes—FIG. 2). The RF energy field is formed by the electrical signals driving the electrodes, wherein the signals are from the generator/controller. The RF field 106 affects the greater occipital nerve 102 and its branches 107.

The greater occipital nerve 102 is a spinal nerve arising between the first and second cervical vertebrae of the spine 104, along with the lesser occipital nerve. It innervates the scalp at the top of the head on the outside of the skull 113. Disorder of this nerve is one of the causes of cervicogenic headaches, referred to as occipital neuralgias. The greater occipital nerve (GON), lesser occipital nerve (LON) and their branches may be referred to as Occipital Nerves (ON). It is understood that an occipital vein 105 can have several branches (not shown) and that the therapeutic RF catheter can be guided into these branches to deliver the therapeutic field to the nerves.

It is understood that the application of Pulsed RF Energy is given as an example of application of energy to nerve tissue. Ablative RF Energy, heat, cold and pulsed stimulation energy can be also used to disrupt, disable or otherwise modulate conduction of pain signals by nerves.

FIG. 2 is a schematic drawing of a portion of an exemplary catheter 108. The distal end of the catheter 103 is equipped with a soft tip 201 and ring electrodes 202. The soft tip can be instrumental in preventing perforation of a blood vessel. Wires connecting the electrodes to the generator can be molded into the catheter shaft (which may be hollow). The shaft may be a plastic, insulting plastic that is biocompatible with a human body. The catheter shaft is preferably flexible to allow navigation through a tortuous blood vessel.

The catheter 108 may incorporate a thermocouple or a thermistor device 207 to sense a temperature of blood adjacent the thermocouple and there by sense the heating effect of the catheter device. During the procedure, the RF current applied by the generator/controller to the electrodes 202 can be controlled to not exceed the desired temperature. Technology to measure blood temperature with a catheter mounted sensor is well known and commercially available. For example, INNERCOOL (Sun Diego, Calif.) manufactures the Accutrol™ Catheter, which measures a patient's core body temperature during therapeutic hypothermia.

FIG. 3 is a schematic diagram of a cross-section along a longitudinal axis of the catheter 108. The hollow lumen 208 may slidably receive a guidewire 204. The guidewire may be used to direct the distal end 110 of the catheter through the jugular vein and occipital veins. Guidewires are frequently used by interventional radiologists to navigate catheters through blood vessels. The pair of electrodes 202a and 202b can be used to apply pulsed RF energy. Electrodes 202a and 202b can be separated by an expandable inflatable balloon 209a. The purpose of the balloon is to direct energy through the nerve tissue and away from blood inside the vessel. Additional electrodes 203, e.g., an annular array of individual electrode pads, can be mounted on the surface of an expandable balloon 209b. For the occipital application it can be expected that suitable balloons can have, for example, expanded diameter of 4-6 mm and length of 5-8 mm. Electrodes, such as electrodes 202a and 202b can have length of 5 mm and spacing between the electrodes can be, for example, 5 to 10 mm. The expansion of the balloon presses the electrodes against the inside wall surfaces of the occipital vein to improve electric contact between the blood vessel walls and the electrodes. Good electrical contact between the electrodes and blood vessel walls is believed to reduce dissipation of RF energy that is intended to be directed to the occipital nerve (ON). Pulsed RF can be applied between any pair of electrodes chosen by the physician. Multiple electrodes and balloons allow for larger area of denervation and can help reduce the time of the procedure, since the exact position of the occipital nerve in relation to the electrodes may not be known. Published United States Patent Application 2005-0288730 to Mark Deem, entitled “Methods and apparatus for renal neuromodulation” discloses several suitable designs of a transvascular catheter adopted for delivery of RF energy. See also, US Published Application, 2006/0142801 entitled “Methods and Apparatus for Intravascularly-Induced Neuromodulation.” The hollow lumen 210 in the catheter may be coupled to a fluid source that forces fluid into the balloon 209a and 209b through apertures 205a and 205b in the distal end of the catheter. Multiple balloons and electrodes may be used to improve the denervation procedure. Electrodes, such as balloon electrodes 203, can be used to perform measurements, such as tissue impedance to enable better control of the procedure and enable the physician to estimate the effect that the application of pulsed RF had on tissue properties.

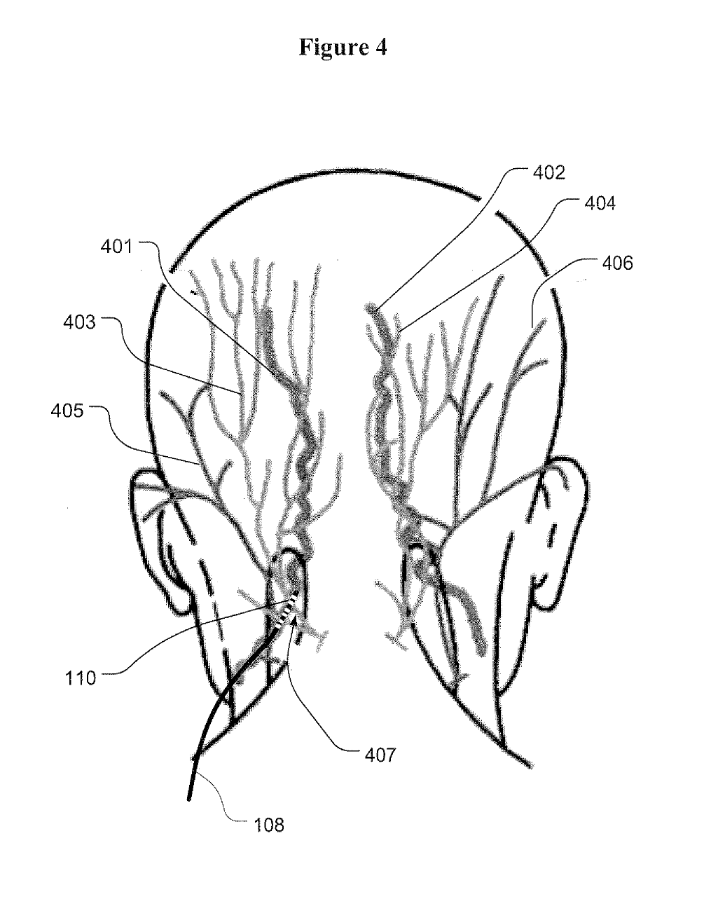

FIG. 4 is an illustration of the back of a skull in which the relative positions of occipital nerves and occipital vessels are illustrated. Left and right occipital arteries 401 and 402 can be seen crossing greater occipital nerves 403 and 404 and minor occipital nerves 405 and 406. These intersection points provide suitable sited for RF energy application, such as for example, location 407 treated with the catheter 108. In the illustrated embodiment the physician advances the distal end 110 of the catheter 108 to a position in the occipital vein that he/she determines is proximate to the occipital nerve. Left greater occipital nerve 403 is shown being treated by the distal end 110 of the catheter 108. Multiple electrodes on the distal end 110 allow broader area coverage at the location 407 where the catheter is expected to cross the path of the greater occipital nerve 405. Since nerves cannot be seen on x-ray, the electrodes (visible on X-ray) are positioned using bony landmarks that indicate where the nerves are usually located. Fluoroscopy (x-ray) or CT (Computer Tomography) is used to identify those bony landmarks. Using radiocontrast injection blood vessels can be illuminated to provide additional landmarks for placement of electrodes.

The electrodes on the distal end 110 of the catheter 108 applies RF energy at location 407, for example. The catheter may include a balloon that presses electrodes against the vessel walls of the occipital veins at a location 407 proximate to the occipital nerves. The proximity needed between the catheter electrodes in the distal end 110 and the occipital nerves is determined by the physician positioning the catheter in the veins and should be sufficiently near such that heating (application of Pulsed RF energy) of tissue near the electrodes results in energy being applied to the occipital nerves. Preferably, the occipital nerves are heated to a temperature. It is believed that loss of nerve function occurs at 60 to 65 degrees Centigrade. Accordingly, a target temperature for the occipital nerve may be 60 to 65 degrees. Higher temperatures, e.g., higher than 65 degrees Centigrade, may be applied by RF to block transmission of nerve signals entirely. However, the pulse RF typically may raises tissue temperatures to only about 42 degrees Centigrade, which does not result in substantial tissue injury, and may sufficiently dull nerve function to provide therapeutic relief from headaches, and especially migraine headaches. It can be expected that the described Pulsed RF procedure will need to be repeated every several months to sustain benefit for headache patients.

The physician actuates the controller/generator 114 to apply a regime of pulsed RF energy to the body tissue proximate to the distal end 110 of the catheter. The application of RF energy heats the tissue. The RF regime can vary depending on parameters that include but are not limited to RF field strength, RF pulse width, the shape of the RF pulse, the catheter tip temperature, the number of pulses and/or the interval between pulses (e.g., duty cycle). Suitable field strengths include, for example, strengths of up to about 10,000 V/cm. Suitable pulse widths include, for example, widths of up to about 1 second. Suitable numbers of pulses include, for example, at least one pulse. Further, pulse may be a pulse burst of, for example two to ten pulses within a short duration, such as one second. The pulses within each burst may have varying amplitudes. Suitable intervals between individual pulses or pulse bursts include, for example, intervals less than about 10 seconds and greater than three seconds. The controller may have one or more pulse RF regimes that are selectively stored in the controller. The regimes may be selected by a physician or preprogrammed into the controller and automatically applied when the physician determines that RF energy is to be applied.

The controller may include limiting controls that, for example, limit the temperature increase in the blood adjacent the catheter and as measured by a thermocouple 207. A suitable catheter target temperature can be for example 40 to 42 degrees Centigrade. An exemplary maximum temperature of the blood adjacent the catheter (as sensed by the thermocouple) may be 45 to 55 degrees Centigrade and an exemplary maximum temperature rise of blood adjacent the catheter may be 0.1 to 0.5 degree Centigrade per second for temperatures above 33 degrees Centigrade (which is approximately body temperature).

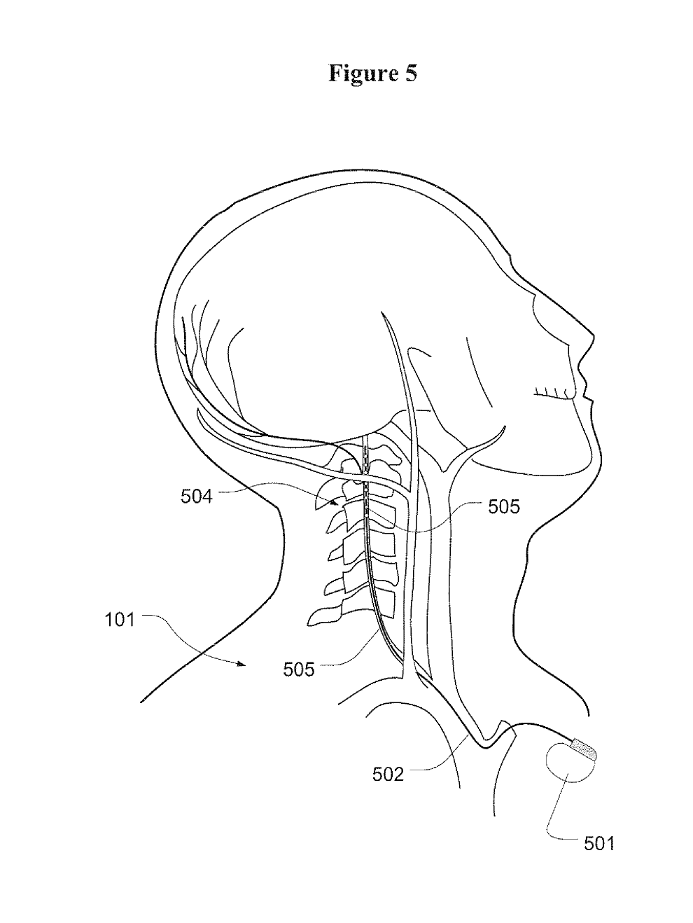

FIG. 5 illustrates a patient 101 treated with one embodiment of an Implanted Pulse Generator (IPG) 501 with an implanted transvascular nerve stimulation lead 502 with electrodes 503 inserted into the vertebral vein 505 to stimulate nerves associated with cervicogenic headaches. Such position of electrodes can be instrumental in stimulating, for example, the dorsal rami of C2 and C3 vertebrae 504 that are known to conduct cervicogenic pain.

Simulators or pulse generators used in this preferred embodiment utilize traditional flexible leads with electrodes. Design and manufacturing of such stimulators is very well understood. A suitable example of an implantable nerve stimulator is the Vagus Nerve Stimulation (VNS™) with the Cyberonics NeuroCybernetic Prosthesis (NCP®) System used for treatment of epilepsy. Other commercially available stimulators are the Genesis Implantable Pulse Generator manufactured by the Advanced Neuromodulation Systems, Inc. (Plano, Tex.) that is used to control pain, and the Medtronic, Inc. (Minneapolis, Minn.) Synergy® Neurostimulation System. These, and many others, state-of-the-art stimulators are fully implantable, externally programmable and operate with a variety of implantable leads and electrodes adapted for long time implantation in the body. With some modifications, stimulators available from Medtronic, Cyberonics and Advanced Neuromodulation Systems can be adapted for this invention. Alternatively, a manufacturing company with right expertise can develop a dedicated stimulator for the invention if the parameters of stimulation are defined.

It is understood that advanced electronic technology and miniaturization allows construction of much smaller “microstimulators”, such as a Bion manufactured by Advanced Bionics of Sylmar, Calif. The Bion's small size allows the entire device to be deployed directly next to the target of stimulation (such as for example a median nerve). Traditional neurostimulation devices consist of an implantable pulse generator (IPG) and electrode lead. Due to the large size of conventional IPGs, this component must be placed away from the site of stimulation in areas such as the chest, abdomen, or buttocks. The electrode lead and often a lengthy extension must then be tunneled under the skin to reach the stimulation site. Implantation of traditional devices involves extensive surgery, sizable scarring, and the possibility of a prominent bulge under the patient's skin. The Bion implantation is a sutureless procedure that uses a set of custom needlelike insertion tools 4 mm in diameter, leaving no visible scar or bulge.

Transvenous Stimulation Experience from Biventricular Pacing (A.K.A. cardiac resynchronization therapy) provides a person skilled in the art with knowledge and expertise in making transvascular stimulation leads. In cardiac resynchronization therapy, an additional lead is placed over the free wall of the left ventricle so that the left and right ventricles are activated simultaneously. Percutaneous placement is now available. The left ventricular lead is placed in one of the branches of the coronary sinus, using one of the commercially available sheath systems. There is no reason to believe that effects and complications of transvenous nerve stimulation using small caliber veins described in this invention will differ significantly from coronary vein (CV) experience.

The use of venous leads for nerve stimulation provides certain advantages over surgical placement of leads that is currently the state of the art. The transvenous access is by far less traumatizing for the patients. Postoperative adhesions and scarring are nearly irrelevant for this mode of stimulation. Increases in electric impedance threshold occur by far less in vein leads than in surgical ones. It is important for preventing postoperative increases in electrical thresholds that leads are securely embedded in their target vein since repetitive chronic vein wall injuries by mobile leads result in progressive fibrotic reorganization of the adjacent vein wall.

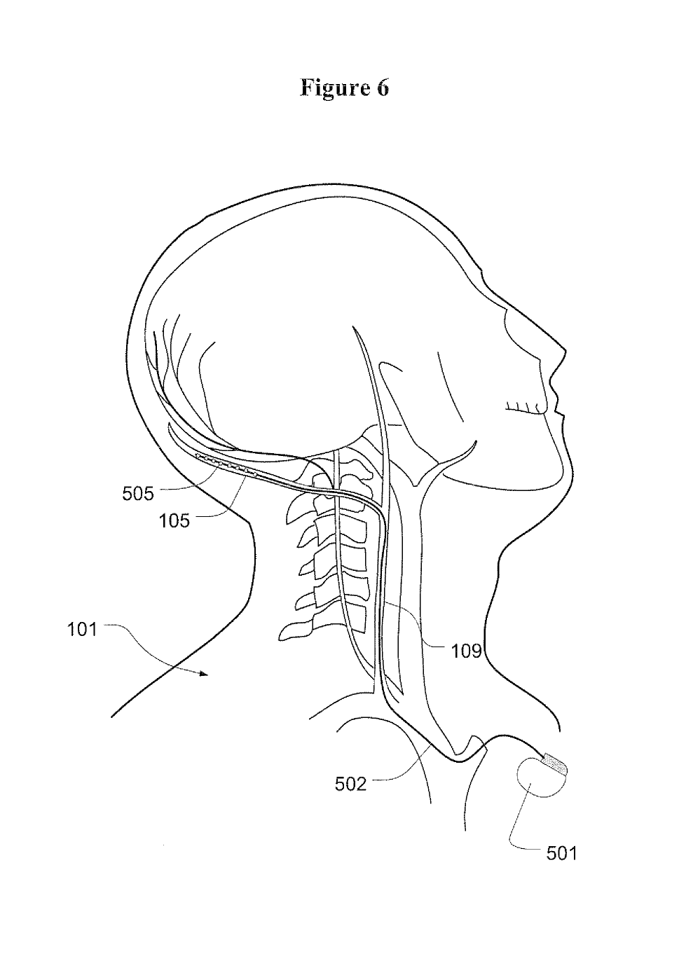

FIG. 6 illustrates a patient 101 treated with another embodiment of an Implanted Pulse Generator (IPG) 501 with an implanted transvascular nerve stimulation lead 502. To position the catheter in the patient and after the catheter tip has been inserted into the jugular vein 109, the electrodes 505 of the lead 501 is advanced into the occipital vein 105 that branches from the jugular vein 109.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method for treating a cervicogenic headache in a human patient having a head and neck, comprising:
inserting a catheter into a vein in the neck or a back of the head of the patient, the catheter having a proximal region, a distal region, and at least one electrode mounted on the distal region;
advancing the catheter into the vein until the distal region is proximal to a peripheral nerve in the neck or head, and
delivering electric energy to distal region of the catheter to applied the energy to the peripheral nerve.

2. The method as in claim 1 where the vein is an occipital vein.

3. The method as in claim 1 where the vein is a vertebral vein.

4. The method as in claim 1 where energy is at least one of pulsed electric field, ablation electric field and thermal energy.

5. The method as in claim 1, where the peripheral nerve is at least one of a dorsal rami of C2 and C3; a superficial medial branch of C3; a C2-C3 zygapophyseal joint; and afferents from cervical nerves C1-C3.

6. The method as in claim 1 wherein the peripheral nerve is heated to at least 60 degrees Centigrade by the delivery of the electrical energy.

7. The method as in claim 6 further comprising minimizing heating of tissue proximate the peripheral nerve by delivering the pulse energy to the peripheral nerve from a catheter tip positioned proximate to the nerve.

8. The method as in claim 6 further comprising monitoring a blood temperature proximate to the peripheral nerve and, if the monitored blood temperature exceeds a predetermined temperature threshold, reducing the amount of delivered electrical energy or ceasing the delivery of electrical energy.

9. The method as in claim 6 further comprising the patient adjusting at least one parameter of the delivery of electrical energy, wherein the patient makes the adjustment based on headaches felt by the patient.

10. A method for treating a cervicogenic headache in a human patient having a neck and head, the method comprising:
implanting a stimulation lead with at least one electrode into a vein in the neck or head of the patient;
where the at least one electrode is positioned in the vein of the patient proximal to a peripheral nerve, and
implanting a pulse generator device in the patient;
electrically coupling the pulse generator to the at least one electrode, and
delivering electric energy from the pulse generator to the electrodes adjacent to the peripheral nerve.

11. The method as in claim 10 where the peripheral nerve is at least one of nerve roots, spinal ganglia, nerve plexus outside a vertebral column or more distal peripheral portion of the targeted nerve, and a nerve in a C1-C3 vertebrae.

12. The method as in claim 11 where the at least one electrode is a plurality of stimulation electrodes within a vertebral vein of the patient.

13. The method as in claim 10 wherein the peripheral nerve is heated to at least 60 degrees Centigrade by the delivery of the electrical energy.

14. The method as in claim 13 further comprising minimizing heating of tissue proximate the peripheral nerve by delivering the pulse energy to the peripheral nerve from a catheter tip positioned proximate to the nerve.

15. The method as in claim 13 further comprising monitoring a blood temperature proximate to the peripheral nerve and, if the monitored blood temperature exceeds a predetermined temperature threshold, reducing the amount of delivered electrical energy or ceasing the delivery of electrical energy.

16. The method as in claim 15 wherein the blood temperature is monitory by a temperature sensor on a catheter positioned in a blood vessel proximate to the peripheral nerve.

17. The method as in claim 13 further comprising the patient adjusting at least one parameter of the delivery of electrical energy, wherein the patient makes the adjustment based on headaches felt by the patient.

18. A method to treat a headache in a human patient having a head and neck, comprising:
positioning a distal section of a catheter in vein in the neck or the head of the patient, wherein the distal section includes at least one electrode;
advancing the catheter into the vein until the at least one electrode is proximal to a peripheral nerve, and
delivering electric energy to the electrode while proximate to the nerve.

19. The method in claim 18 further comprising heating the peripheral nerve using the energy delivered to the at least one electrode.

20. The method in claim 18 wherein the vein is an occipital or vertebral vein.
